(19) United States
(12) Patent Application Publication
(10) Pub. No.: US 2018/0092839 A1
(43) Pub. Date: Apr. 5, 2018

(54) MEDICATED SPRAY FOR TREATMENT OF SUBSTANCE ABUSE, OVERDOSE, ADDICTION AND IMPULSE CONTROL DISORDERS

(71) Applicant: Lance L. GOOBERMAN, Merchantville, NJ (US)

(72) Inventor: Lance L. GOOBERMAN, Merchantville, NJ (US)

(21) Appl. No.: 15/283,751

(22) Filed: Oct. 3, 2016

(51) Int. Cl. A61K 9/08; A61K 31/485; A61K 9/00

(52) U.S. Cl. CPC A61K 9/08; A61K 9/006; A61K 9/0043; A61K 31/485

(57) ABSTRACT

Provided herein are portable application devices and compositions for nasal and oral delivery of substances and compositions to treat overdose, addiction and/or behavioral disordered persons.

### FIELD OF THE INVENTION

The present invention is directed to portable application devices and compositions for nasal and oral delivery of substances and/or compositions to treat substance overdose, addiction and/or behavioral disordered persons, and particularly as an effective on demand apparatus, composition and method to treat possible substance abuse overdoses and/or to provide a readily available antidote to various substances which are or may oftentimes prove dangerous, and potentially fatal.

### BACKGROUND OF THE INVENTION

Many substance and/or behavioral addicted persons have experienced much difficulty with impulse control and oftentimes engage in unhealthy or unproductive self medication, some of which may be prescribed but yet ineffective or impractical, For example, there are both nicotine substitutes/replacements available without a prescription and replacements which do require a prescription. These include lozenges, patches, gum, sprays and electronic cigarettes or other simulated smoking devices.

Heroin use and addiction has become a major problem approaching epidemic proportions in recent times, compounded by the availability of synthetic opiates many times more potent than heroin leading to an unprecedented number of overdoses and deaths.

Many heroin treatment regimens are also problematic including, for example, substitute medications for opiates, such as heroin or Oxycontin®, include Buprenorphine, sold under the brands of Suboxone® and Subutex® and methadone available as Dolophine® Methadose® and Methadone Diskets®. Methadone is a tightly controlled substance available only from licensed methadone clinics, whereas Buprenorphine is available by prescription at many pharmacies. Buprenorphine is a controlled substance and only available through specifically licensed entities.

The goal on many of the substitutes and replacements for the original substance is to provide a replacement that is not nearly as enjoyable as the original, but one that prevents highly unpleasant withdrawal symptoms from occurring. For example, Buprenorphine is a partial opiate antagonist, or otherwise possess a drug or medication efficacy which stimulates activity of opiate receptors in the brain, but yet does not produce as strong an effect as a full opiate agonist such as morphine, methadone, oxycodone, hydrocodone, heroin and codeine. Like full opioid agonists, dissimulation occurs at receptors which are normally stimulated by naturally occurring opioids called endorphins. Buprenorphine by displacing agonists from receptors and preventing re-attachment and has the effect of reducing withdrawal symptoms and suppressing cravings, yet it does not have the same strong pleasure characteristic of faster acting opiate drugs Buprenorphine also has a longer withdrawal period than other shorter acting opiates. Because Buprenorphine has a very long half life or the half life of how long it takes the body to break down and eliminate the presence of a drug, it remains in the system relatively longer than other agonists and does not need to be taken as frequently which has the benefit of helping to reduce cravings, suppress unpleasant withdrawal symptoms and break the habit of a quicker more repetitive administration of a faster acting opiate drug or the abuse of such. Notwithstanding, buprenorphine itself often becomes an addictive problem.

Methadone, on the other hand, is a full opiate agonist and mimics the pharmacological activity of opiates such as heroin. Methadone differs from heroin in its pharmacokinetics in that it is an oral medication that must pass through the digestive track slowing its excess to the brain relative to heroin but lasting much longer than heroin. In this way methadone is useful for preventing withdrawal symptoms and is thought to allow people to taper their heroin use down gradually or their methadone use down gradually without producing the expected sought after more extreme high associated with a faster acting opiate agonist. However, as with buprenorphine, methadone is also a problem addictive compound due, inter alia, to a long half-life.

As to further examples of addiction treatment, nicotine replacement therapy (NRT) comes in several forms. Skin patches (Habitrol® and Nicoderm®), gum (Nicorette®), and lozenges (Commit®) deliver controlled doses of nicotine, These delivery methods are available over the counter (OTC), without prescription. The nasal spray and inhaler are available by prescription (Nicotrol®). These products replace nicotine but eliminate the toxins in tobacco combustion products. Many generic products for NRT are also available.

Another known method is to attempt to restore healthy neurophysiological functioning such so that people do not use addictive substances or activities as a means for self-medication, or to correct neurophysiological damage resulting from chronic drug use, or addictive activities.

Acamprosate (Camprol®) is used to treat alcohol abuse, and is also known to decrease some of the physical and psychological symptoms associated with alcohol withdrawl. Acamprosate is believed to restore the chemical balance in a brain that became unbalanced by chronic alcohol use. While remaining an FDA approved medication, recent studies now doubt its effectiveness.

Buproprion (Zyban®, Wellbutrin® Voxra®, and Budeprion®) are drugs that are used to target relapse prevention for tobacco withdrawal. Nicotine is the addictive drug in tobacco products like cigarettes, cigars, and chewing tobacco. This medication is in the class of atypical antidepressants. These medications block the reuptake (reabsorption) of the neurotransmitters dopamine and norepinephrine. Reuptake blockers work by allowing neurotransmitters to remain in the synapse for a longer period. This permits them to bind to more receptors. Re-establishing activity in neurotransmitter systems enhances the recovery process. Bupropion, originally used as an antidepressant, has lately been used in a compound called Contrave®, a combination of bupropion and naltrexone, and is used to treat obesity stemming from, inter alia, food and/or carbohydrate addiction.

Still other example medications contemplated for use herein are: Exonatide (Byetta® and Bydureon®) an injectable used to treat diabetes, and liraglutide (Victoza® and Saxenda®) also used in diabetes treatment in addition to other dulaglutides such as Trulicity®.

Varenicline (Chantix®) is also used to treat tobacco use disorder or addiction, and is known as a partial agonist of the nicotine receptor. It's unclear exactly how this compound works, but appears to suppress cravings associated with nicotine use by stimulating the brain's reward system. Natrexone is also known to treat nicotine use by its effects on the dopaminergic system.

Benzodiazepines (e.g. Xanax®, Librium®, Ativan®, Klonopin®, Diazepam/Valium®) are often called anti-anxiety drugs or anxiolytics, and are thought by many as the most controversial in addiction treatment as many people become addicted to these drugs as they are frequently abused (see Sedative, Hypnotic, or Anxiolytic Use Disorders). This class of drugs works by activating the GABA receptors in the brain to relieve the anxiety associated with withdrawal. They also can compensate for changes that occur in the GABA system following withdrawal from sedative drugs such as alcohol or opiates. Furthermore, some people may abuse alcohol or opiates because they are attempting to self medicate a pre-existing anxiety disorder. Anti-anxiety drugs arc also useful for treating these underlying disorders. Anxiety is also a symptom of many other types of psychiatric disorders. To some, the cautious and judicious use of benzodiazepines may he helpful, particularly during the early stages of recovery, but addiction to benzodiazepines, as mentioned, remains a concern.

Conventional treatment also includes the prevention or diminishment of powerful cravings that cause people to resume drug use or an addictive activity, after a period of cessation.

Naltrexone has several recognized uses, and in some applications may be useful for those aiming to moderate alcohol consumption, and/or to treat sex addiction, gambling urges, internet pornography, pedophilia, and any and all other activities that involve the dopaminergic system, or the so-called “reward” brain system. One of naltrexone's known primary functions is to suppress alcohol craving. In other applications, a preferred embodiment herein, naltrexone and other opiate antagonists, such as a naloxone, are useful in controlling opiate use urges, and even more importantly in combating potential overdoes.

Thus, to this end there is seen a need for the convenient and sometimes spontaneous application by nasal and/or oral delivery of substances and compositions to treat or self treat as needed substance addicted and/or behavioral disordered persons on demand, and possible substance abuse overdoses.

### SUMMARY OF THE INVENTION

In its broadest aspect, the present invention provides apparatus and compositions in the form of a spray applicator for administering nasally or orally an anti-addiction and/or behavioral treatment and/or overdose treatment or prevention substance selected from any of the above described substances in the background of the invention, and/or any other such known (or yet unknown) anti-addiction and/or behavioral treatment and/or overdose treatment or prevention substances, and in preferred embodiments an opiate antagonist selected from naloxone and/or naltrexone. The applicator is capable of delivering metered, calibrated single or multiple doses of the anti-addiction and/or behavioral treatment and/or overdose treatment or prevention substance, such as an opiate antagonist, on demand through a projecting delivery portion which is shaped or dimensioned for introduction into the nose or mouth. A pharmaceutical composition for nasal or oral administration is also disclosed which may comprise, for example, an addiction antagonist and/or behavioral treatment and/or overdose treatment or prevention substance, preferably an opioid antagonist such as naloxone and/or naltrexone, and which may comprise a water-susceptible solid carrier admixed with the opioid antagonist.

### DETAILED DISCLOSURE

This invention provides compositions for application by spray nasally or orally in the treatment and/or reversal of substance abuse and addiction, overdose and/or treatment of behavioral disorders, such as addiction to opiates and opioids, including heroin, opium, oxycontin, oxycodone, morphine, methodone and tramadol, addiction to prescription drugs such as benzodiazepines, including vallum and xanax, amphetamines, hypnotics, barbitunates, cocaine, methamphetamine, PCP and other addictive and/or behavioral disorders, such as gambling urges and tendencies and sex and pornography addiction, opioid depression, alcohol abuse, self-injurious behaviors, such as self mutilation, kleptomania, and other known other impulse control disorders treatable by agonists and/or antagonists in accordance with the invention.

Additional examples of compounds on substances contemplated as treatable by the invention as an antidote include the following, sometimes referred to as designer opiates and/or opioids, including, but not limited to, 3-Methylbutyrfentanyl, 3-MBF, 3-Methylfentanyl, 3-MF, 4-Chloroisobutyrfentanyl, 4-CliBF, p-CliBF, 4-Fluorobutyrfentanyl, 4-FBF, p-FBF, 4-Fluoroisovutyrfentanyl, p-FiBF, 4-Methoxybutyrfentanyl, 4-MeO-BF, p-MeO-BF, 4-Fluorofentanyl, 4-FF, p-FF, Acetylfentanyl, AF, Acrylfentanyl, AD-1211, AH-7921, α-Methylfentanyl, “China White”, Butyrfentanyl, BF, Desmethylprodine, MPPP, Furanylfentanyl, Fu-F, 4′-Nitromethopholine, MT-45, Nortilidine, O-Desmethyltramadol, U-5175^{[62]}, U-47700, U-77891, Valerylfentanyl, VF, W-15^{[63]}, W-18.

In a preferred embodiment, apparatus for applications and compositions are provided for buccal or nasal administration for treatment of patients suffering from opiate and/or opioid over-dosage.

With respect to a preferred aspect treatment of the invention addicts of opiates and opioids such as heroin and methadone sometimes suffer respiratory failure as a result of administration of an excessive dose of the drug. While opiate antagonists may be given to reverse severe opiate respiratory depression, the standard method of administration is by intravenous injection, which is difficult for a medically unskilled person to carry out successfully, particularly in the stress of an emergency situation. For simplicity of discussion, reference herein to opiate antagonists shall mean both opiate and opioid antagonists.

In a preferred aspect, the present invention seeks to provide systems of administering an opiate antagonist which can be carried out by an unskilled person rapidly and with a good chance of successfully reviving a patient suffering from opiate over-dosage. The compositions of the invention have the advantage that they can be administered by a first-aider or person having no medical training, such as a friend or neighbor of an addict. A single dose of the antagonist can readily be sprayed into the nose or mouth of an addict who is having difficulty breathing, while undertaking standard resuscitation procedures. If the patient does not respond to the initial dose, further doses of the antagonist can be given until reversal of the opioid and/or opiate depression is apparent. An advantage is that treatment can be given quickly and effectively without the need for the first aider to find a blood vessel and give an intravenous injection. Another advantage of the applicators of the invention is that they cannot he misused to give injections of other drugs and are thus more likely to be retained and used for their intended purpose.

The present invention may be embodied in many other specific forms employing any one or more of the pharmaceutically active, or active pharmaceutical ingredients (“API”) or bioactive substances and/or agents mentioned herein above to combat and/or treat substance addictive and/or behavioral disorder persons, impulse and urges including a myriad of suitable and/or effective application apparatus therefor without departing from the spirit or essential attributes thereof, and may include any known, and as yet unknown, substances and/or agents for combating and treating substance addicted and/or behavioral disordered persons.

According to one aspect of the present invention there is provided a spray applicator having a solution of an opiate and/or opioid antagonist selected from naloxone and/or naltrexone provide in a reservoir. The applicator is designed to be capable of delivering single or multiple doses of an efficacious amount of the antagonist from the reservoir, and the applicator portion preferably comprises a projecting delivery portion shaped and dimensioned for introduction into the nose or mouth of a patient.

According to another aspect of the invention there is provided a pharmaceutical composition for oral or nasal administration comprising an opiate and/or opioid antagonist, the composition being comprised in finely-divided solid form and comprising a water-susceptible solid carrier and the antagonist compound.

In a further preferred embodiment, the spray applicator may be designed for dispensing the solution into the mouth, e.g. sub-lingually, and be provided with a projecting delivery portion for this purpose. An additionally preferred embodiment, the applicator is provided with a delivery portion which is shaped and dimensioned for introduction into a nostril so that the dose is sprayed directly into the nasal passages, and which method of administration may be more convenient and enables resuscitation to be continuously and simultaneously applied. Such a device which has such a projecting delivery portion is also advantageous it may be applied directly into the mouth. Suitable spray applicators may be single trip devices, and normally incorporate a pump or syringe action for forcing an amount of the solution of the antagonist compound out of a nozzle, or calibrated to administer a premeasured amount of antagonist.

According to the aspect of the invention in which the pharmaceutical composition is in powder form, it is preferably administered nasally. In this embodiment, the composition is packaged via a dispenser having a projecting portion for introduction into a nostril, and preferably in calibrated measured amounts. Normally, a propellant is employed for generating an aerosol of the powdered pharmaceutical in a stream of gas. The dispenser will generally include calibration means for metering doses of the composition dispensed into the patient's nasal passages.

Preferred opiate and/or opioid antagonists for use in the compositions of this invention include naloxone (17-allyl-6-deoxy-7,8-dihydro-14-hydroxy-6-oxo-17-normorphine), and naltrexone, (17-(cyclopropylmethyl)-4,5α-epoxy-3, 14-djntydroxymorphinan-6-one,), although any efficacious antagonist and/or agonist is contemplated for use in this invention, or any anti-addiction and efficacious behavioral disorder treatment substance in any effective treatment combination is contemplated for use herein.

Naloxone has a high affinity for u-opioid receptors in the central nervous system, and is a u-receptor competitive antagonist, and a pure antagonist with no agonist properties. Naloxone has long been used to counter the effects of opiate overdose, such as heroin or morphine, and specifically life threatening depression of the central nervous system, respiratory system, and is also used to treat hypotension. Nalaxone is also combined with buprenorphine in a drug composition called Suboxone which is used to treat opiate addiction.

Enteral naloxone has also been used with opiate therapy in mechanically ventilated acute care patients in the reduction of gastritis and esophagitis. Additionally, a combination of oxycodone and naloxone has been used for the prophylaxis of opiate induced constipation in patients requiring strong opiate therapy, and known as Targin and Targinact. A variant of naloxone known as (+)-nalaxone has also been used in treating opiate-related addiction, in binding to TLR4 immune receptors and inhibiting production of dopamine responsible for substance addiction, but still retaining pain relieving effect. For example, in the simultaneous administration of morphine and (+)-nalaxone, the intended analgesic effect of morphine will be present but without the potential for morphine addiction.

Naltrexone is an opiate receptor antagonist with qualitatively different effects than naloxone, and is most commonly known as useful in dependence treatment, rather than in emergency overdose treatment in reversibly blocking and attenuating the effects of opiates. Using naloxone in place of naltrexone is known to cause acute opiate withdrawal symptoms, and conversely using naltrexone in place of naloxone in a possible overdose situation is known to possibly lead to insufficient opiate antagonism and failure to reverse the overdose. Naltrexone is primarily known to help patients overcome opiate addiction by blocking euphoric effects. Naltrexone is also well known in its effective treatment of alcohol dependence and for its efficacy in reducing frequency and severity of relapse to drinking. Naltrexone also has been shown to reduce heavy drinking when used in people who continue drinking while taking naltrexone, and may even have increased efficacy in alcohol consumption treatment when used during active drinking rather than during abstinence, known as the Sinclair Method. Other known uses for naltrexone include treatment of depersonalization disorder, and in low doses (“low-dose naltrexone, or LDN”) used for treating non-chemical dependency maladies such as multiple sclerosis, fibromyalgia, and even forms of cancer and HIV, and in self-injurious behaviors, often times present in persons with developmental disabilities, such as autism, to inhibit the release of beta-endorphin which binds to the same receptors as opiates, such as heroin and morphine. Natrexone is also known to be useful in treatment of impulse control disorders, such as compulsive gambling, theft or kleptomania, pornography addiction and compulsive hair pulling, or trichotillomenia.

In some preferred embodiments herein a mixture of two or more anti-addiction or behavioral disorder treatment substances, such as two or more opiate antagonists may (or opiate or opiate/opioid antagonists, as the case may be) be employed. In the use of antagonists, preferably, naloxone may be used as a spray-able liquid composition and naltrexone may he used in the form of a powdered, solid composition, usually for nasal administration, such as in a spray particulate mist application along with sprayable liquid use as well. Naltrexone in a spray form in a dosage range as discussed herein is preferred as a deployable spray/atomized delivery herein for several reasons, although any substance abuse treatment compound or urge treatment compound in any physical mode is contemplated for use herein.

Naltrexone in a spray form in a dosage range as disclosed herein is preferred for use due to its longer duration of efficacy and longer potency. This compound when administered will not wear off quickly, and will most likely not have to be repeatedly administered such as the less potent naltrexone charges, and, for example, naloxone currently in wide spread conventional use. A dose of naltrexone, compared to naloxone, is preferred due to the greater potency of naltrexone. Naltrexone is also preferably used herein to precipitate withdrawal such that a patient may be treated with additional naltrexone in various forms, such as in implanted pellet form or an injectable form, to maintain abstinence.

Notwithstanding, any drug compound that is absorbed subcutaneously and/or any medication that can be administered subcutaneously is contemplated for use herein as a spray administered compound including, adrenaline, and/or epinephrine, as a non-selective adrenaline agonist to potentially reverse the effects off allergens as well as substances of abuse and/or an adverse reaction to any compound.

It has been found, surprisingly, in accordance with one aspect of this limitation that it is possible to administer naltrexone in the form of liquid solution at low doses but yet higher than conventionally taught by the nasal and/or buccal route with excellent results both for blocking and/or the reversal of undesirable side-effects due to the administration of opioids and in the case of excessive ingestion thereof.

Low dose according to the invention means a dose above 1% (w/v) and preferably 3-5 mg/injection.

Thus, the liquid formulations for nasal administration according to the invention contain amounts of naltrexone (normally in the form of hydrochloride salt) greater than about 1.0% (w/v), and preferably about 3-5 mg/injection.

As used herein, the terms “about” or “approximately” broaden the numerical value. For example, in some cases, “about” or “approximately” refers to +/−10%, of the relevant unit value. Also, the disclosure of ranges is intended as a continuous range including every value between the minimum and maximum values recited.

As used herein, the term “treatment” embraces all the different forms or modes of treatment as known to those of the pertinent art and in particular includes preventative, curative, delay of progression and palliative treatment.

Unless otherwise specified, values expressed as % refer to % w/v.

As used herein, the term “opioid receptor antagonist” includes any substance that selectively blocks an opioid receptor of any type (e.g., mu, delta, kappa, etc.) or subtyped (e.g., mu1/mu2). Suitable opioid receptor antagonists for use in the present invention include, but are not limited to, any centrally acting opioid receptor antagonist. In some embodiments, the antagonist is selected from naltrexone, nalmefene, naloxone, naloxonazine, nor-binaltorphimine, the opioid receptor antagonist is naltrexone.

The term “subject”, or “patient”, refers to an animal, for example, a mammal, such as a human, who is the object of treatment. The patient may also be a domestic production animal, exotic zoo animal, wild animal, or companion animal. The subject, or patient, may be either male or female.

Opioid receptor agonists (sometimes abbreviated as opioid agonists, or opioids) and opioid receptor antagonists (sometimes abbreviated as opioid antagonists) can also be called opiates.

The opioid receptor antagonist may be in free form or in pharmaceutically acceptable salt or complex form.

The composition is administered transmucosally to the patient. In some embodiments, the transmucosal administrations is selected from intranasal, buccal, sublingual, vaginal, ocular and rectal route of administration. In one specific embodiment, the composition is administered sublingually to the patient. In a preferred embodiment, the composition is administered intranasally to the patient.

The liquid solutions according to the invention are normally aqueous solutions or aqueous-alcoholic solutions in which the alcohol is preferably ethanol, and preferably in an amount to deliver a dosage amount greater than 1 mg, and preferably about 3-5 mg per administration. In addition, the solutions may contain a buffer, the purpose of which is to maintain the pH at the value at which the opioid antagonist is in the form of a salt, for example as hydrochloride. The buffers may be selected from the following: citric acid/sodium citrate, citric acid/sodium hydroxide, dibasic sodium phosphate/citric acid, dibasic sodium phosphate/monobasic potassium phosphate, acetic acid/sodium acetate. The excipients used for the compositions of this type may also comprise antimicrobial preservatives, agents that increase the tonicity and agents that increase the viscosity of the solution (viscosity improvers). Oil based compositions and various suspensions, as known in the art are also contemplated for use herein.

Among the antimicrobial preservatives, such compounds may include, for example, benzalkonium chloride, methylparaben, propylparaben, sodium benzoate, benzoic acid, phenylethyl alcohol or mixtures thereof, such as, for example, in amounts between 0.005-0.50% (w/v), preferably 0.005-0.30% (w/v), more preferably 0.01-0.1% (w/v).

Agents that increase tonicity are, for example, sodium chloride, dextrose, lactose or mixtures thereof, such as, for example, in amounts between 0.1-5.0% (w/v), preferably 0.1-2.0% 0 (w/v), more preferably 0.1-0.9% (w/v).

Viscosity improvers may be selected from, for example, hydroxypropyl methylcellulose (hypromellose), hydroxyethyl cellulose, hydroxypropyl cellulose, methylcellulose, microcrystalline cellulose, carboxymethylcellulose sodium, xanthan gum or mixtures thereof, such as, for example, in amounts between: 0.01-2.0% (w/v), preferably 0.02-1.0% (w/v), more preferably 0.05-0.5% (w/v).

The formulations according to the invention may be, by example, prepared following the standard techniques employed for preparing solutions for nasal application.

The following may be, for example, dissolved in a given amount of water: the preservative, the salts for the buffer, the agent for increasing osmolality, and then the viscosity improver. When the solution obtained is clear, the active ingredient is dissolved therein and the solution made up to the required volume with water.

Where the antagonist is in the form of a liquid composition, it may be a solution in a pharmaceutically acceptable carrier or co-solvent such as water or an alcohol, such as ethanol e.g. an aqueous solution containing an appropriate percentage of ethanol. Naloxone and naltrexone are both freely soluble in water and aqueous alcohol when in the form of a salt, such as a hydrochloride. Alternatively, the opiate antagonist may be dissolved in dilute saline solution, e.g. approximately isotonic salt solution. The composition may include a buffering agent to maintain the opiate in solution in the salt form, e.g. a phosphate buffer, such as sodium hydrogen phosphate to maintain the solution at a slightly acidic pH. A solution of the antagonist, usually in the form of the hydrochloride, for example, at a dosage amount greater than 1 mg, preferably 3-5 mg per administration in spray form be employed for nasal or buccal administration in accordance herein. The liquid composition may be packaged in a calibrated metered dosage spray dispenser, using a pump or propellant.

In the case of a solid, powdered composition for nasal administration, the antagonist is mixed with one or more solid, powdered carriers. Suitable carriers include saccharides such as sorbitol, mannitol, lactose, fructose, glucose and sucrose. Other carriers include water-soluble or swellable polymers such as cellulose derivatives, for example, hydroxypropyl methyl cellulose and carboxymethyl cellulose. A solid salt of the antagonist, e.g. the hydrochloride, maybe mixed with a carrier, or coated with the carrier or with a third material such as a hydrophilic polymer.

The solid, powdered composition containing the opiate antagonist may be packaged in a dispenser with a suitable propellant, such as HFC-134a or HFC-227. Again, a valve may be provided, which is adapted to dispense a dosage unit of the antagonist, or medication of about greater than 1 mg per administration preferably 3-5 mg per administration in accordance with this invention.

It may also be desirable to include an anti-oxidant, such as ascorbic acid or citric acid in the powdered formulation.

The compositions of the invention may also include antiparkinson compounds or drugs to treat or relieve the effects of Parkinson's disease like symptoms including, but not limited to, L-Dopa, Deprenyl, Tyrosine Hydroxylase, Apomorphine, and Anticholinergic drugs.

The compositions of the invention may also include any one or more of common additives, adjuvants, excipients, coloring agents, thickeners and the like commonly found in any pharmaceutical formulation or composition and/or found in neutraceutical compositions.

The invention is illustrated by the following Examples of pharmaceutical compositions suitable for use in dispensing an opiate antagonist in accordance with the invention and by the accompanying drawing, and description of one form of spray applicator in accordance with a preferred embodiment of the invention for dispensing a liquid composition.

### Example 1

Sprayable aqueous liquid composition for a nasal applicator.

Naloxone hydrochloride was dissolved in a solution of purified water to form a solution containing 0.8% weight/volume of the naloxone. Benzalkonium chloride was added to the hydrochloride solution in an amount of 0.025% weight/volume as a preservative. The solution may be buffered to a pH of about 6.5 using a phosphate buffer (sodium or potassium hydrogen phosphate). The solution was packaged into a dispenser as shown in the accompanying drawing, giving a shot volume of 50 μl (micro litre) which is equivalent to a unit dose of 400 μg (microgram) per shot.

### Example 2

Solid, powdered nasal preparation.

Powdered solid naloxone hydrochloride was mixed with powdered dextrose or lactose in an amount of from 2% weight/volume naloxone HCl and 98% weight/volume of the finely powdered sugar. The resulting mixture can be subsequently coated with a vinyl pyrollidone to form a free-flowing powder in which the opioid antagonist is present in a concentration of 2% by weight. The powdered composition is packaged in a dispenser, for example, as described in WO 99/27920.

### Example 3

Naloxone HCl was dissolved in water with mannitol or lactose in a weight ratio of 2:98. The resulting solution was spray dried or freeze dried to form a fine powder containing 2% of naloxone HCl.

The powdered product can he packaged in an aerosol can with a low boiling propellant fitted with a metering valve or in a dispenser as described, for example, in WO 99/27920.

In some embodiments, a kit may be provided and comprise one or more single dose containers filled with the active pharmaceutical ingredient (“API”) composition, a sheet of instructions, and one or more mucosal atomization devices (MADs). in some embodiments, one MAD is pre-fitted to each single dose container. in some embodiments, the kit comprises one or more MADs capable of being fitted to the single dose container(s) prior to use. In some embodiments, the MAD can be fitted to a syringe, or an MAD with syringe can be used in conjunction with a filled vial. Any MAD capable of being fitted to a syringe, e.g., fitted with a luer lock, can be employed. MADs are available commercially and include LMA/MAD Nasal™ intranaslamucosal atomization device (LMA North America, Inc., Sand Diego, Calif.) and Wolfe-Tory Mucosal Atomization Device MAD (Wolfe-Tory Medical, Salt Lake City, Utah).

In an additional example device, an applicator may comprise a body part moulded from a flexible plastic material and having a projecting part suitably sized for insertion into a nostril. The projecting part has an internal tube, which extends from a tip to approximately a junction between the projecting part and a main body part. At its distal end, a tube may be joined to the inside of the projecting part, e.g. by forming part of an integral moulding, and communicate with a discharge orifice. A solution of the drug or API compound to be dispensed is contained in reservoir which is preferably made from transparent plastic or glass so that it can be seen by inspection if it contains any API or approximately how much API it does contain. For this purpose, the solution may be colored with a pharmaceutically acceptable dye.

A piston is made from flexible plastic material (e.g. polythene) and carries a solid piston rod which is formed with a passage. The passage communicates with the interior of the reservoir and terminates in a cross bore. The assembly consisting of the reservoir and piston and piston rod may he fitted into the body of the applicator by introducing the rod into the tube. The rod is a free fit into the part of the tube nearest to main body part but is a tighter fit into the distal end of the tube. With the projection parts situated in the patient's nostril, pressure is applied to the free end of the reservoir, e.g. by placing the fingers on the surfaces of the device and the thumb on the end of the reservoir and squeezing. This forces liquid with API from the reservoir along a passage, out of the cross bore and into the tube. Continued pressure forces liquid in a spray out of the orifice by the rod acting as a piston in the tube. The tube may be tapered slightly towards the orifice so that higher pressure can be developed within its distal end. It will be appreciated that by shaping the projecting part as a tapering fit in the nostril, a major amount of the API composition is retained in the nasal passages or within the mouth.

Although the invention herein has been described with reference to particular embodiments, it is to be understood that these embodiments are for illustrative purposes only. The present invention may be embodied in many other forms employing any of the pharmaceutical active agents or API's mentioned herein without departing from the spirit and scope of the invention as defined in the appended claims.

1. A pharmaceutical formulation in the form of liquid solution for spray administration by the nasal and/or buccal route containing naltrexone and/or nalmefene as the active ingredient in amounts greater than 1% effective to block and/or reverse physiological effects of mixtures of opiates and opioids, including heroin and fentanyl and/or one or more fentanyl analogs.

2. Formulation according to claim 1, characterized in that the naltrexone and/or nalmefene is in an amount of about between 3-5 mg/injection.

3. Formulations according to claim 1, wherein said liquid solutions are aqueous or aqueous-alcoholic solutions.

4. Formulations according to claim 1, wherein said formulations also comprise a buffer selected from the group consisting of: citric acid/sodium citrate, citric acid/sodium hydroxide, dibasic sodium phosphate, citric acid, dibasic sodium phosphate/monobasic potassium phosphate, and acetic acid/sodium acetate buffer.

5. Formulations according to claim 4 additionally containing: antimicrobial preservatives, agents that increase the tonicity and agents that increase the viscosity of the solution.

6. Method for the administration of a formulation according to claim 1, characterized in that said formulations are administered in the form of spray.

7. A portable spray application device containing the formulation of claim 1 to treat substance addicted and/or behavioral disordered persons.

8. (canceled)

9. A portable spray application device containing the formulation of claim 1 for delivery of naltrexone and/or nalmefene in treatment of potential opioid overdose.

10. The device of claim 1 for treatment of potential respiratory failure.

11. The device of claim 2 for treatment of potential respiratory failure.

12. The device of claim 3 for treatment of potential respiratory failure,

13. The device of claim 4 for treatment of potential respiratory failure.

14. The device of claim 5 for treatment of potential respiratory failure.

15. The device of claim 6 for treatment of potential respiratory failure.

16. The device of claim 7 for treatment of potential respiratory failure.

17. The device of claim 8 for treatment of potential respiratory failure.

18. The device of claim 9 for treatment of potential respiratory failure.
